(19) United States
(12) Patent Application Publication
(10) Pub. No.: US 2018/0103899 A1
(43) Pub. Date: Apr. 19, 2018

(54) MICRO-STRAIN SENSOR FOR IMPLANTABLE DEVICES

(71) Applicant: INTERNATIONAL BUSINESS MACHINES CORPORATION, Armonk, NY (US)

(72) Inventors: Amos Cahan, Dobbs Ferry, NY (US); Katsuyuki Sakuma, Fishkill, NY (US)

(21) Appl. No.: 15/296,382

(22) Filed: Oct. 18, 2016

(51) Int. Cl. A61B 5/00; G01L 1/22

(52) U.S. Cl. CPC A61B 5/4851; G01L 1/2262; A61F 2/82; A61B 5/0022; A61B 2562/0261; A61B 5/686

(57) ABSTRACT

An apparatus for monitoring strain caused by bodily fluids or tissues in contact with an implantable medical device includes at least one strain gauge sensor embedded within the implantable medical device. The strain gauge sensor is configured to measure a mechanical strain of the implantable medical device. The apparatus further includes a processor module coupled with the strain gauge sensor. The processor module is configured to receive a sense signal generated by the strain gauge sensor and to extract therefrom a measurement of strain caused by bodily fluids or tissues in contact with the implantable medical device.

### BACKGROUND

The present disclosure relates generally to electronic sensors, and more particularly relates to sensors used for biomedical applications.

In a biomedical context, a stent is a tubular support structure placed temporarily or permanently into the lumen of an anatomic vessel or duct which is used to keep the passageway open, often to facilitate healing or relieve an obstruction, such as in coronary arteries. A graft, similar in function to a stent, is a tubular-shaped structure which is permanently implanted into the lumen of an anatomic vessel or duct. Stents and grafts are part of an important treatment modality used both to remedy narrowing or occlusion of the arterial lumen (e.g., in coronary artery disease or renal artery stenosis) as well as to replace a malfunctioning vessel wall because of a disease in the vessel wall (e.g., aortic aneurysm). Vascular stents are usually comprised of metal and vascular grafts are commonly comprised of Dacron, polyurethane nanofabric, or the like. A shunt (e.g., ventriculoperitoneal shunt) is a small hole or passage which moves, or allows movement of, fluid from one part of the body to another. Shunts are often used as conduits for bodily fluids.

Narrowing of the lumen of a stent or graft may follow implantation. This may be due, at least in part, to proliferation of tissue surrounding the stent or graft (e.g., restenosis). Narrowing of the lumen of a stent or graft may also result from the formation of a blood clot on the surface of a stent or graft facing the vessel lumen (e.g., in-stent thrombosis). Patients with a coronary artery, renal artery or brain stent are usually prescribed medications to prevent platelet aggregation (e.g. aspirin, clopidogrel, or the like). Narrowing of the lumen of a stent or graft may further be caused by an embolus from another part of the body that cannot pass through the stent or graft, said embolus being composed of a blood clot, microorganisms, or fat. A shunt may fail due to dislocation, kink or obstruction. All of these conditions will result in reduced flow within the device.

Noninvasive measurement of blood flow across an intravascular device is traditionally inaccurate and requires special equipment and expertise which are not readily available. Occlusion of a coronary artery by in-stent thrombosis may result in sudden cardiac death from a myocardial infarction (i.e., heart attack) or a fatal arrhythmia. Occlusion of a stent elsewhere (e.g., a cerebral artery) can cause ischemia and infarction of tissues supplied by the vessel in which the stent had been implanted. Likewise, failure of a ventriculoperitoneal shunt may cause a reduced level of consciousness, hydrocephalus and increased intracranial pressure. Monitoring of blood flow across stents and/or grafts in a continuous, noninvasive manner is clinically invaluable as it can inform clinicians on the hemodynamic state of a patient.

### SUMMARY

Aspects according to one or more embodiments of the present invention relate to a micro-scale semiconductor or metal strain gage embedded or integrated with an implantable medical device adapted to be inserted within a lumen (i.e., a cavity or channel within a tube or tubular organ) through which bodily fluids flow to monitor flow and/or to detect a malfunction of the implantable device leading to impaired flow.

In one aspect, the invention relates to an apparatus for monitoring the strain caused by bodily fluids in contact with an implantable medical device, the apparatus including at least one strain gauge sensor embedded within the implantable medical device. The strain gauge sensor is configured to measure a mechanical strain of the implantable medical device. The apparatus further includes a processor module coupled with the strain gauge sensor. The processor module is configured to receive a sense signal generated by the strain gauge sensor and to extract therefrom a measurement of strain caused by bodily fluids or tissues in contact with the implantable medical device.

In a further aspect, the invention provides a method for monitoring the strain caused by bodily fluids or tissues in contact with an implantable medical device. The method includes: embedding a strain gauge sensor with the implantable medical device, the strain gauge sensor generating a sense signal indicative of a strain caused by bodily fluids or tissues in contact with the implantable medical device; extracting from the sense signal generated by the strain gauge sensor a measurement of the strain caused by bodily fluids or tissues in contact with the implantable medical device; and determining an estimate of a flow of the bodily fluids through the implantable medical device as a function of the measurement of the strain.

Techniques as disclosed herein can provide substantial beneficial technical effects. By way of example only and without limitation, one or more embodiments may provide one or more of the following advantages:
intermittent, on-demand, or continuous monitoring of one or more functions of an implantable device;
quantitative or qualitative estimation of flow within an implantable device by means of monitoring strain on its walls;
early detection of subacute or acute compromise in the effective diameter of the lumen of an implantable device;
early detection of implantable device malfunction due to dislocation, kink or dehiscence, among other factors;
indirect assessment of cardiac output and blood pressure, blood volume and/or blood viscosity, pressure in compartments of the heart, and pressure in the pulmonary circulation; and
monitoring of heart rate, pulse velocity and/or respiratory rate.

These and other features and advantages will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIGS. 1A through 1E are top plan views conceptually depicting several configurations of illustrative metallic foil strain gauges which can be used in conjunction with embodiments of the present invention;

FIGS. 2A through 2C are top plan views depicting at least a portion of different configurations of illustrative semiconductor strain gauges which are well-suited for use in embodiments of the invention;

FIG. 3A is a schematic diagram depicting at least a portion of an exemplary strain sensor circuit, according to an embodiment of the present invention;

FIG. 3B is a schematic diagram depicting at least a portion of an exemplary dual-gauge strain sensor circuit, according to an embodiment of the present invention;

FIG. 3C is a conceptual view depicting a stent including two strain sensors placed at opposite ends of the stent for detecting the movement upstream and downstream of a blood vessel and measurement of the flow of blood through the stent, according to an illustrative embodiment of the invention;

FIG. 3D is a schematic diagram depicting at least a portion of an exemplary four-gauge strain sensor circuit, according to an embodiment of the invention;

FIG. 4 is a block diagram conceptually depicting at least a portion of an exemplary strain sensor system, according to an embodiment of the invention;

FIG. 5 is a schematic diagram depicting at least a portion of an exemplary strain sensor system including wireless transmission capabilities, according to an embodiment of the invention;

FIG. 6 is a block diagram conceptually depicting at least a portion of an exemplary strain sensor system including multiple sensor elements, according to an embodiment of the invention;

FIGS. 7A and 7B conceptually depict an application of a stent with attached strain sensor, according to an embodiment of the invention; and

FIG. 8 conceptually depicts an illustrative strain sensor system in which one or more components of the system are attached to the outside surface of the skin, according to an embodiment of the invention.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

Principles of the present disclosure will be described herein in the context of illustrative apparatus, systems and methods for estimating the flow and/or pressure of bodily fluids or tissues across a portion of a lumen (i.e., a cavity or channel within a tube or tubular organ) or a compartment of the heart through which bodily fluids flow. More particularly, one or more embodiments provide apparatus, systems and methods for monitoring the strain caused by bodily fluids or tissues in contact with an implantable medical device and micro-scale semiconductor or metal strain gauges embedded or integrated with an implantable medical device adapted to be inserted in a lumen (i.e., a cavity or channel within a tube or tubular organ) or compartment through which bodily fluids flow to monitor flow and/or to detect a malfunction of the implantable device leading to impaired flow. The inventive apparatus, systems and methods, in one or more embodiments, are configurable to perform operator-independent, continuous monitoring of strain through one or more miniaturized transmitter-sensor equipped strain gauge sensors embedded in or integrated with implantable medical devices, such as, but not limited to, vascular stents/grafts, venous filters, prosthetic heart valves, artificial hearts, bone implants, shunts, etc. It is to be appreciated, however, that the specific apparatus, systems and/or methods illustratively shown and described herein are to be considered exemplary as opposed to limiting. Moreover, it will become apparent to those skilled in the art given the teachings herein that numerous modifications can be made to the embodiments shown that are within the scope of the appended claims. That is, no limitations with respect to the embodiments shown and described herein are intended or should be inferred.

A strain gauge is a well-known device for measuring the strain (i.e., measure of deformation representing a displacement between particles in an object relative to a reference length on the object). Strain gauges are adapted to measure strain that can be correlated to stress, force, torque, and various other stimuli, including displacement, acceleration and position. The flow and/or pressure of bodily fluids and/or tissues in contact with the sensor directly or indirectly can be extracted from the strain measurement and monitored over time. This information can be used to detect a process compromising the functioning of the implantable device to which the strain gauge is attached, such as, for example, blood clotting, infection, inflammation or tissue proliferation leading to restenosis of the lumen, or other mechanical malfunction of the implantable device (e.g., kink or dislocation). In the illustrative case of prosthetic heart valves or aortic grafts, pressure across the valve can be measured continuously to thereby monitor device function.

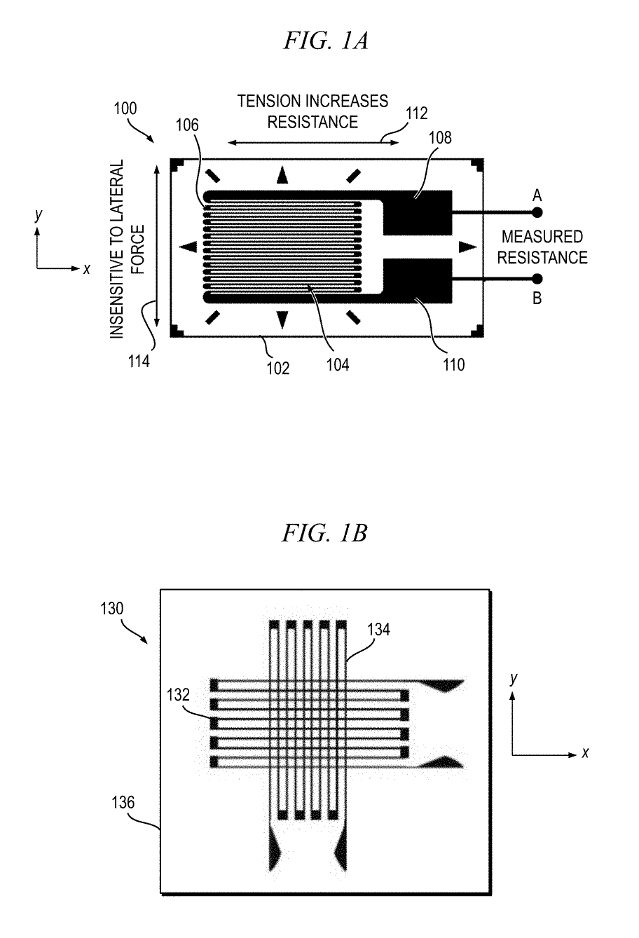

By way of example only and without limitation, FIGS. 1A-1E are top plan views conceptually depicting several configurations of illustrative metallic foil strain gauges which can be used in conjunction with embodiments of the present invention. With reference to FIG. 1A, a basic illustrative strain gauge 100 is depicted that includes an insulating flexible backing 102 which supports a metallic foil pattern 104. The metallic foil pattern 104, in this embodiment, is comprised of a continuous conductive track or trace 106, arranged in a serpentine (i.e., winding) pattern to increase an effective resistance of the track, connected with a pad or terminal, 108 or 110, at each end of the conductive track. Embodiments of the invention are not limited to any particular configuration, dimensions or material of the strain gauge.

A resistance of the strain gauge 100 is measured across two nodes, A and B, electrically connected to the two terminals 108 and 110, respectively, of the strain gauge. The strain gauge 100 is attached to an object to be measured by a suitable attachment means, including, but not limited to, using adhesive (e.g., epoxy, cyanoacrylate, or the like), using a designated adapter to secure the strain gauge to an object to be monitored, or connected to the object using other techniques (e.g. soldering, welding, etc.). As the object to which the strain gauge is attached is deformed, the metallic foil pattern 104 will also deform, thereby causing a change in electrical resistance of the strain gauge 100, as measured between nodes A and B. This resistance change, usually measured using a resistance bridge, such as a Wheatstone bridge, or alternative detection circuitry, is related to the strain by a quantity known as the gauge factor. Generally, the strain gauge 100 is configured to measure tension (i.e., strain) in a direction parallel to a major axis (x-axis) of the metallic foil pattern 104 (i.e., in a dimension substantially parallel to a direction of the conductive track). Furthermore, the strain gauge 100 is highly insensitive to lateral force in a direction parallel to a minor axis (y-axis) of the strain gauge (i.e., in a dimension substantially perpendicular to a direction of the conductive track). In the embodiment shown, the conductive track 106 is comprised primarily of constantan, manganin, or similar materials in the case of a metallic strain gauge. In one or more embodiments, the width of a wire turn (metal foil) for a metallic strain gauge is about 15 μm and the thickness of a wire (metal foil) for metallic strain gauge is about 3 although embodiments are not limited to any specific dimensions or types of materials used to form the strain gauge 100.

FIGS. 1B through 1E are top plan views conceptually depicting several illustrative configurations of multiple-element strain gauges which can be used in conjunction with embodiments of the present invention. With reference to FIG. 1B, a multi-element strain gauge 130 includes first and second metallic foil strain gauge elements, 132 and 134, respectively, supported on a flexible insulating substrate 136. The second strain gauge element 134 is formed overlying the first strain gauge element 132 (i.e., in a different plane), with the two strain gauge elements being oriented perpendicular to each other; that is, the first and second strain gauge elements are rotated 90 degrees relative to one another. A flexible insulating layer (not explicitly shown, but implied) resides between the two strain gauge elements 132, 134 so that they are electrically isolated from one another. The strain gauge 130 formed in this manner is adapted to detect strain in both x and y dimensions.

FIG. 1C depicts at least a portion of an illustrative multi-element strain gauge 140 which includes first and second metallic foil strain gauge elements, 142 and 144, respectively, supported on a flexible insulating substrate 146. In this embodiment, the first and second strain gauge elements 142, 144 are formed adjacent to one another in a same plane (i.e., non-overlapping), with the two strain gauge elements being oriented perpendicular to one another. Thus, like the strain gauge 130 shown in FIG. 1B, the strain gauge 140 is capable of detecting strain in both x and y dimensions, although the amount of area (i.e., footprint) required for the strain gauge 140 is greater than the strain gauge 130.

FIG. 1D depicts at least a portion of an illustrative multi-element strain gauge 150 which includes first, second and third metallic foil strain gauge elements, 152, 154 and 156, respectively, supported on a flexible insulating substrate 158. In this embodiment, the first, second and third strain gauge elements 152, 154, 156 are formed adjacent to one another in a same plane (i.e., non-overlapping), with the three strain gauge elements being shifted θ=120 degrees relative to one another. It is to be appreciated that embodiments of the invention are not limited to any specific number of strain gauge elements or offset angle (θ) between the respective strain gauge elements.

Similarly, FIG. 1E depicts at least a portion of an illustrative multi-element strain gauge 160 which includes first, second and third metallic foil strain gauge elements, 162, 164 and 166, respectively, supported on a flexible insulating substrate 168. In this embodiment, the first, second and third strain gauge elements 162, 164, 166 are formed overlying one another (i.e., in different planes), with the three strain gauge elements being shifted 120 degrees with respect to each other. A flexible insulating layer (not explicitly shown, but implied) resides between each pair of strain gauge elements lying in adjacent planes so that all strain gauge elements are electrically isolated from one another. The more strain gauge elements that are employed, the more directions the strain can be detected, although this enhanced accuracy comes at the expense of increased space, complexity and circuitry, among other tradeoffs.

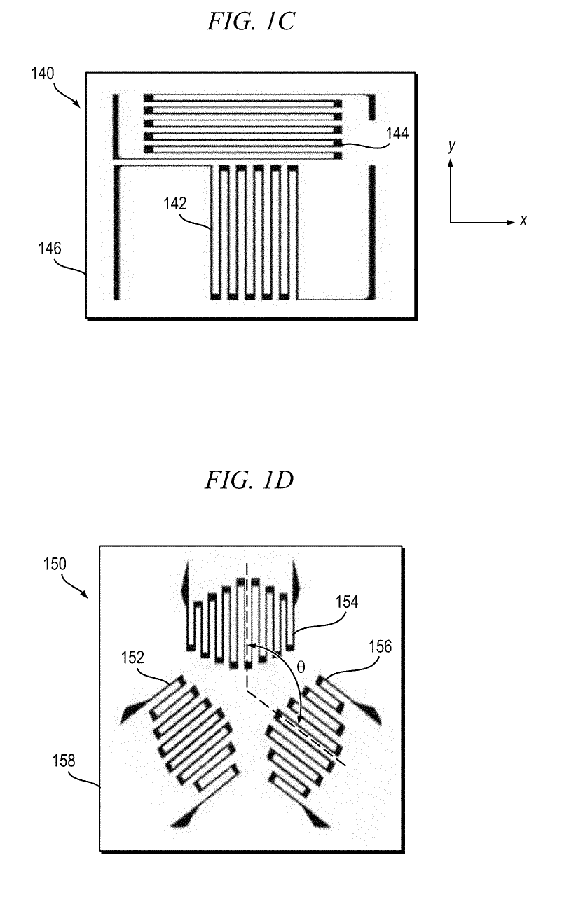

In one or more embodiments, a semiconductor material (e.g., doped silicon) is utilized for the strain gauge, as shown in FIGS. 2A-2C by way of example only. Specifically, FIGS. 2A-2C are top plan views depicting at least a portion of different configurations of illustrative semiconductor strain gauges which are well-suited for use in embodiments of the invention. As previously stated, semiconductor strain gauges often employ doped silicon as the material forming a resistive element of the strain gauge, between the two strain gauge terminals (e.g., 108 and 110 in FIG. 1A), although embodiments of the invention are not limited to doped silicon. FIG. 2A depicts an exemplary semiconductor strain gauge 200 having an I-shaped resistive element 202, FIG. 2B depicts an exemplary semiconductor strain gauge 210 having a U-shaped resistive element 212, and FIG. 2C depicts an exemplary semiconductor strain gauge 220 having a W-shaped resistive element 222. A resistance of the resistive elements 202, 212, 222 of the semiconductor strain gauges 200, 210, 220, respectively, can be controlled as a function of one or more characteristics of the strain gauges, including, for example, the type of semiconductor material, doping level of the material, dimensions (e.g., length and width) of the resistive elements, and shape of the resistive elements, as will become apparent to those skilled in the art. In a stent application, an I-shaped strain gauge (e.g., 200 shown in FIG. 2A) may be preferred, since space is very limited and an I-shaped semiconductor strain gauge offers one of the smallest footprints compared to other strain gauge configurations.

As previously stated, the gauge factor defines the relationship between the change in resistance of a strain gauge and the strain to which the gauge is subjected. Table 1 below lists gauge factors, γ, for different illustrative materials and their corresponding nominal compositions (by weight percentage, wt %). For a given strain gauge, the gauge factor is a constant. As apparent from Table 1, The gauge factor for metal strain gauges (e.g., metallic foil) generally ranges between 0.45 and 2.2; the gauge factor for semiconductor materials is considerably higher than for metals (e.g., up to about 200). In forming a semiconductor strain gauge, a base semiconductor material (e.g., silicon) is preferably doped, such as by diffusion of an impurity or dopant (e.g., boron for p-type, or arsenic or phosphorous for n-type) of a prescribed doping level into the base semiconductor material, to obtain a base resistance as desired.

TABLE 1

| Material | Nominal Composition, wt % | Gauge Factor, γ |
|---|---|---|
| Constantans | 55 to 65 Cu, 35 to 45 Ni | 1.8 to 2.2 |
| Manganins | 10 to 15 Mn, 1.4 to 4.5 Ni, Fe, rem | 0.45 to 0.55 |
| | Cu | |
| Nickel-chromes | 20 Cr, 0 to 25 Fe, rem Ni | ~1.9 |
| Nickels | . . . | ~−12 |
| Silicons* | . . . | ±110 to ±200 |

*Includes varying degrees of impurities present

The change in conductivity of semiconductor materials is generally larger than for metals. Furthermore, the size of a semiconductor strain gauge is typically smaller compared to metallic strain gauges, thus making semiconductor (e.g., doped silicon) strain gauges more suitable for use with implantable devices. For example, in one or more embodiments, the width (W) of an exemplary semiconductor strain gauge (e.g., 200 in FIG. 2A) is about 200 μm and the length (L) is about 2 mm, although embodiments are not limited to any specific dimensions of the strain gauge. Accordingly, there is a tradeoff between gauge size and change in conductivity which should be considered depending on the type of application in which the strain gauge is to be employed. The gauge factor, y, is defined according to expression (1) as follows:

$$γ = \frac{Δ R / {R}_{G}}{ϵ} , \qquad ( 1 )$$

where ΔR represents the change in resistance (i.e., conductivity) caused by an applied strain, R_{G} represents the resistance of the undeformed gauge (i.e., with no strain), and ϵ represents strain. Thus, assuming the resistance R_{G} of the undeformed strain gauge and the gauge factor γ are known, the strain ϵ can be determined from the measured change in resistance ΔR of the strain gauge using expression (1). As apparent from expression (1), the strain gauge relationship is linear.

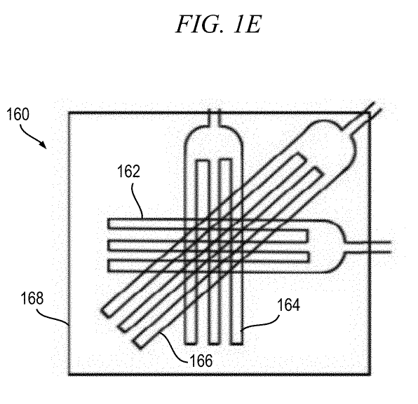

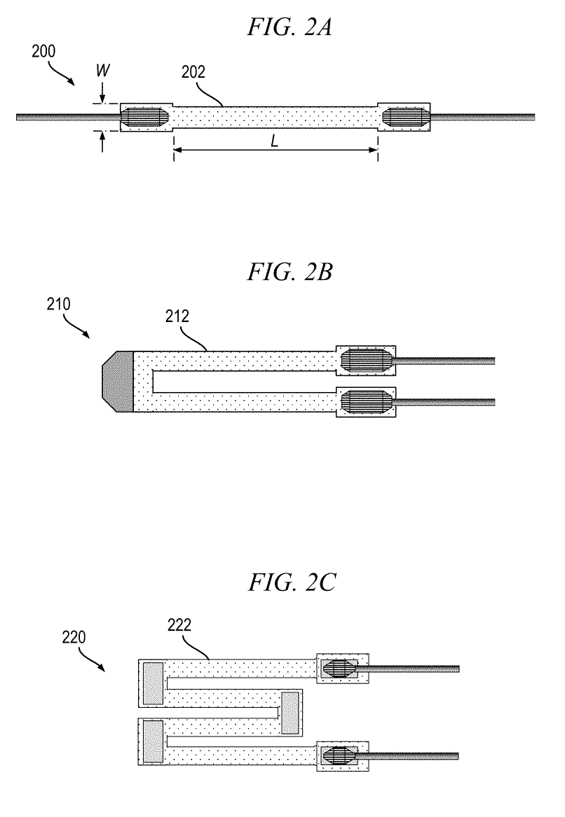

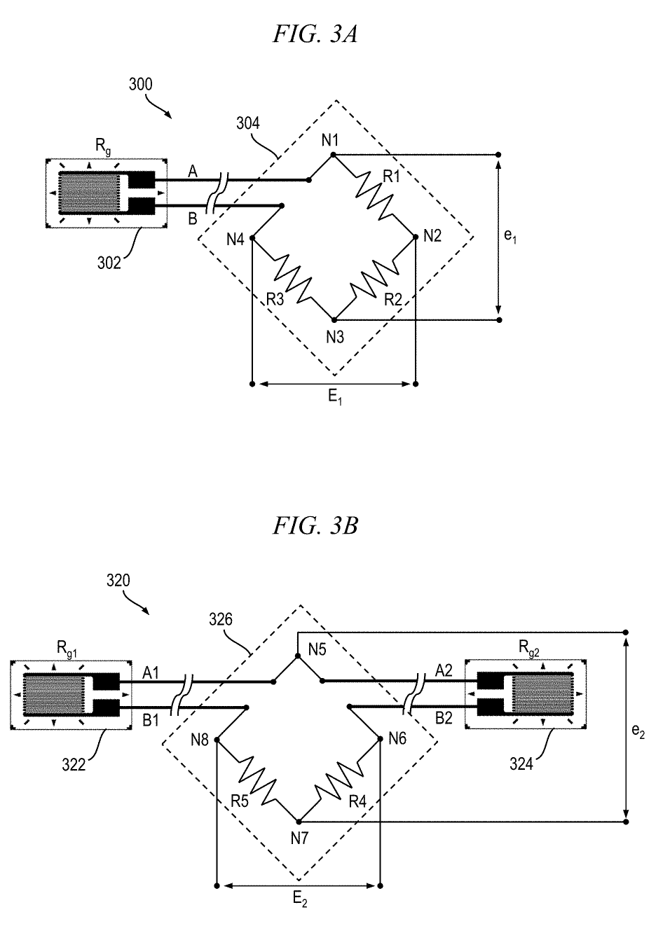

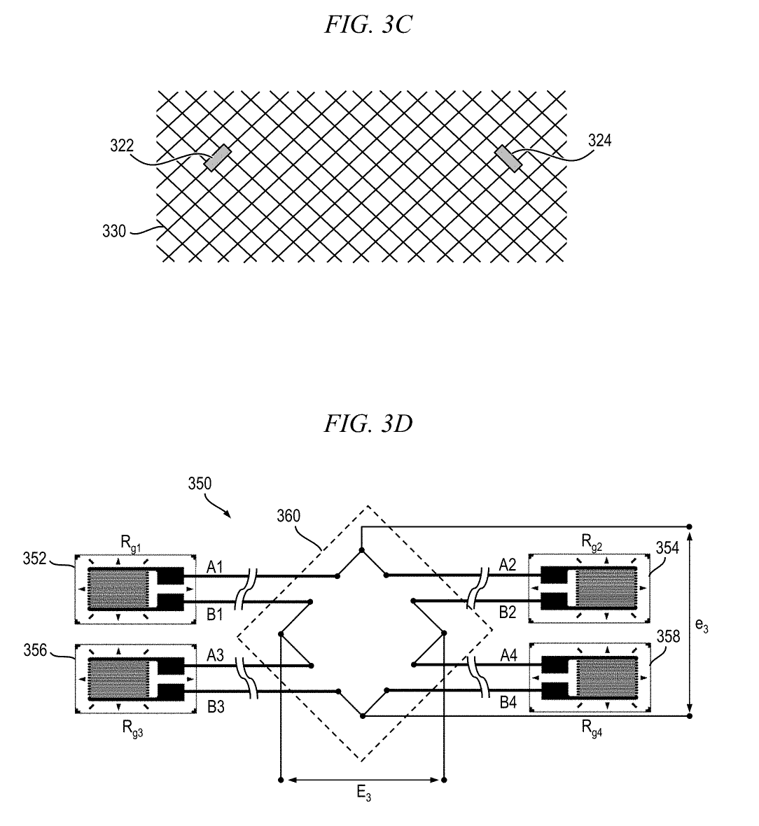

FIG. 3A is a schematic diagram depicting at least a portion of an exemplary strain sensor circuit 300, according to an embodiment of the invention. The strain sensor circuit 300 includes a strain gauge 302, which may be a metallic foil or semiconductor strain gauge, connected with a detection circuit 304, which in this embodiment is configured as a Wheatstone bridge or other resistance bridge. As previously stated, other detection circuitry configured to measure the change in resistance (ΔR) of the strain gauge 302 may be similarly employed, according to embodiments of the invention.

In this illustrative embodiment, the strain gauge 302, having a resistance R_{g}, replaces one of the resistors in the Wheatstone bridge 304. Specifically, a first terminal (A) of the strain gauge 302 is connected to a first terminal of a first resistor, R1, in the Wheatstone bridge 304 at a first node N1. A second terminal of resistor R1 is connected to a first terminal of a second resistor, R2, in the Wheatstone bridge 304 at a second node N2. A second terminal of resistor R2 is connected to a first terminal of a third resistor, R3, in the Wheatstone bridge 304 at a third node N3. A second terminal of resistor R3 is connected to a second terminal (B) of the strain gauge 302 at a fourth node N4.

As will be known by those skilled in the art, a Wheatstone bridge is an electrical circuit used to measure an unknown electrical resistance by balancing two legs of a resistance bridge circuit, one leg of which includes a component whose resistance is to be determined; in this case, the resistance R_{g} of the strain gauge 302. The remaining resistors R1, R2 and R3 are assumed to be known. A primary benefit of a Wheatstone bridge is its ability to provide extremely accurate measurements. In one or more embodiments, the resistors R1, R2 and R3 in the Wheatstone bridge 304 are configured to be equal to a resistance, R_{g}, of the undeformed strain gauge 302. An output voltage, e₁, of the strain sensor circuit 300, measured between nodes N1 and N3 of the circuit, is determined according to expression (2) as follows:

$${e}_{1} = \frac{{E}_{1}}{4} \cdot γ \cdot {ɛ}_{0} , \qquad ( 2 )$$

where E₁ represents an excitation voltage applied between nodes N2 and N4 of the strain sensor circuit 300, γ is the gauge factor of the strain gauge 302, and ε₀ represents strain of the test piece. The measured output voltage e₁ correlates directly to a resistance of the strain gauge 302, which can then be used to determine the amount of strain using expression (1) above.

FIG. 3B is a schematic diagram depicting at least a portion of an exemplary dual-gauge strain sensor circuit 320, according to another embodiment of the invention. The strain sensor circuit 320 includes first and second strain gauges, 322 and 324, respectively, which may be metallic or semiconductor strain gauges, for example, connected with a detection circuit 326, which in this embodiment is configured as a Wheatstone bridge. The first and second strain gauges 322, 324 replace corresponding resistors in the Wheatstone bridge 326. Specifically, a first terminal (A1) of the first strain gauge 322 is connected to a first terminal (A2) of the second strain gauge 324 at a first node N5. A second terminal (B2) of the second strain gauge 324 is connected to a first terminal of a first resistor, R4, in the Wheatstone bridge 326 at a second node N6. A second terminal of resistor R4 is connected to a first terminal of a second resistor, R5, in the Wheatstone bridge 326 at a third node N7. A second terminal of resistor R5 is connected to a second terminal (B1) of the first strain gauge 322 at a fourth node N8. In one or more embodiments, the resistors R4 and R5 in the Wheatstone bridge 326 are configured to be equal to a resistance, R_{g1}, of the undeformed first strain gauge 322, which is configured to be equal to a resistance, R_{g2}, of the undeformed second strain gauge 324. An output voltage, e1, of the strain sensor circuit 320, measured between nodes N5 and N7 of the circuit, is determined according to expression (3) as follows:

$${e}_{2} = \frac{( {ɛ}_{1} - {ɛ}_{2} ) \cdot {E}_{2}}{4} \cdot γ , \qquad ( 3 )$$

where E₂ represents an excitation voltage applied between nodes N6 and N8 of the strain sensor circuit 320, γ is the gauge factor of the strain gauge 322 or 324 (which are preferably identical to one another), ε₁ represents strain of the first strain gauge 322, and ε₂ represents strain of the second strain gauge 324.

The sensor circuit 320 will detect the difference of two strains using this dual strain gauge configuration. For example, as shown in FIG. 3C, by detecting different timings of deformation of two individual strain gauges 322 and 324 placed at opposite ends of a stent 330, the movement upstream and downstream of a blood vessel and the flow of the blood can be measured, which may be helpful in determining the condition of the blood vessel.

The two strain gauges 322, 324 can be connected to two individual Wheatstone bridge circuits to thereby determine the deformation timing and deformation at each location. Alternatively, the two strain gauges 322, 324 can be connected to a single Wheatstone bridge circuit and the amount of strain detected by each strain gauge summed. More than two strain gauges can be employed in different locations if desired, in accordance with one or more embodiments of the invention. By connecting two to four individual strain gauges to the same Wheatstone bridge circuit, each strain can be added or subtracted based on the configuration; one of the configurations is shown in FIG. 3B to which expression (3) above relates. In one or more other embodiments, all four resistances in the Wheatstone bridge circuit can be replaced with strain gauges to further enhance the sensitivity of the sensor circuit, as in the exemplary strain sensor circuit shown in FIG. 3D, according to an embodiment of the invention. More particularly, FIG. 3D depicts an exemplary strain sensor circuit 350 comprising four strain gauges, 352, 354, 356 and 358, connected to the same Wheatstone bridge circuit 360. By using this method, the required space for the Wheatstone bridge circuit, amplifier circuits, etc., can be beneficially reduced. While this circuit arrangement is rarely used for strain measurement, it is often used for strain gauge transducers. When the strain gauges 352, 354, 356, 358 at the four sides of the Wheatstone bridge circuit 360 have their respective resistances changed to R_{g1}+ΔR_{g1}, R_{g2}+ΔR_{g2}, R_{g3}+ΔR_{g3}, and R_{g4}+ΔR_{g4}, the output voltage e₃ can be determined according to expression (4) or (5) as follows:

$$\begin{matrix}{e}_{3} = \frac{1}{4} ( \frac{Δ {R}_{g 1}}{{R}_{g 1}} - \frac{Δ {R}_{g 2}}{{R}_{g 2}} + \frac{Δ {R}_{g 3}}{{R}_{g 3}} - \frac{Δ {R}_{g 4}}{{R}_{g 4}} ) \cdot {E}_{3} , or & ( 4 ) \\ {e}_{3} = \frac{1}{4} \cdot K ( {ɛ}_{1} - {ɛ}_{2} + {ɛ}_{3} - {ɛ}_{4} ) \cdot {E}_{3} , & ( 5 )\end{matrix}$$

where K is the gauge factor, E₃ represents an excitation voltage applied to the strain sensor circuit 350, ε₁ represents strain of the first strain gauge 352, ε₂ represents strain of the second strain gauge 354, ε₃ represents strain of the third strain gauge 356, and ε₄ represents strain of the fourth strain gauge 358.

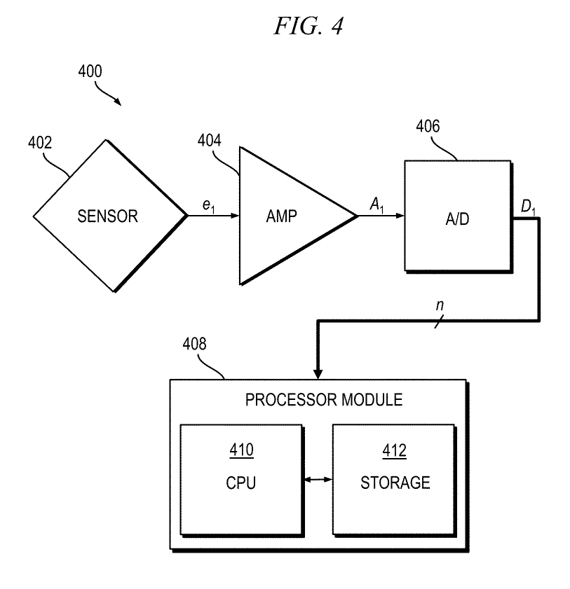

FIG. 4 is a block diagram conceptually depicting at least a portion of an exemplary strain sensor system 400, according to an embodiment of the invention. The system 400 includes a sensor circuit 402, which in one or more embodiments is implemented in a manner consistent with any of the illustrative strain sensor circuits 300, 320 and 350 shown in FIGS. 3A, 3B and 3D, respectively. The system 400 includes an amplifier (Amp) circuit 404, which in one or more embodiments may be implemented using an operational amplifier or the like. A sensed output signal, e₁, generated by the sensor circuit 402 is supplied to an input of the amplifier 404. This output signal e₁ is, in one or more embodiments, a voltage indicative of a change in resistance of a strain gauge included in the sensor circuit. This output voltage is correlated to a strain imposed on the sensor circuit 402, which may be determined using expression (2), (3), (4) or (5) above, as previously stated.

The amplifier 404 is configured to receive the output signal e₁ and to generate an analog amplified sense signal, A₁, having an amplitude which is a function of a gain of the amplifier. Although shown as a separate functional module, the amplifier 404 may, in one or more embodiments, be integrated with the sensor 402. The amplified sense signal A₁ is supplied to an input of an analog-to-digital (A/D) converter 406 which is configured to generate a digital output signal, D₁, which is an n-bit digital representation of the amplified sense signal A₁, where n is an integer. The digital output signal D₁ is supplied to a processor module 408 which, in one or more embodiments, includes a central processing unit (CPU) 410, or alternative processor, operatively coupled with a storage element 412 (e.g., a memory). The CPU 410 is configured to receive the digital signal D₁ and to determine, as a function thereof, a strain imposed on the sensor circuit 402 as a function of the digital signal. In one or more embodiments, the processor module 408 may be configured to extract strain information directly or indirectly from the analog sense signal e₁ generated by the sensor 402. In such an embodiment, the amplifier 404 and the A/D converter 406 can be omitted from the strain sensor system 400, and the output of the sensor 402 supplied directly to the processor module 408.

As previously explained, the flow and/or pressure of bodily fluids in contact with the sensor 402 directly or indirectly can be extracted from the strain measurement. The digital signal D₁ indicative of the strain measurement(s) can be stored in the memory 412 for subsequent processing and/or for collecting a history of strain associated with an implantable medical device (e.g., stent) utilizing the novel sensor circuit, according to one or more aspects of the invention.

It is to be appreciated that one or more functional components of the strain sensor system 400 may reside externally to the human body. For example, the sensor 402, amplifier 404 and A/D converter 406 may reside within the human body, attached to the implantable device, and the processor module 408 may reside externally (i.e., outside the body), in accordance with one or more embodiments. In another embodiment, the sensor 402, amplifier 404, A/D converter 406, CPU 410 may reside within the human body, attached to the implantable device, and a second processor module (configured to communicate with the CPU 410) may reside externally (i.e., outside the body). Alternatively, the sensor 402 and amplifier 404 may reside within the body and the A/D converter 406 and processor module 408 may reside outside the body. In order to accomplish this, a wireless communication link is established so that signals generated by the sensor 402 within the body are transmitted outside the body for further processing. Various other combinations and arrangements of the functional components of the strain sensor system 400 are similarly contemplated, according to embodiments of the invention.

For example, in one or more embodiments the processor module 408 may be adapted to receive the digital output signal, D₁, via a wireless communication link (e.g., infrared, Bluetooth, ultrasound, Wi-Fi, radio frequency identification (RFID), optical, near-field communication (NFC), ANT+, etc.) established between the A/D converter 406 and the processor module. In this arrangement, a transmitter may be included in the system 400 having an input coupled with the output of the A/D converter 406, and having an output coupled with an antenna for transmitting the digital signal D₁ wirelessly to a corresponding receiver associated with the processor module 408. Suitable transmission circuitry for wirelessly transferring data between the A/D converter 406 and the processor module 408 will become apparent to those skilled in the art given the teachings herein.

If the strain sensor system 400 does not include a battery, the system will preferably include energy harvesting circuitry that can be integrated with the implantable device or, in one or more embodiments, a near-field communication (NFC) protocol or the like can be used to send power to the device wirelessly. In this latter approach, the strain sensor system 400 may include a power receiver for receiving an energy signal and extracting therefrom a supply voltage or current suitable for powering the circuitry in the strain sensor system.

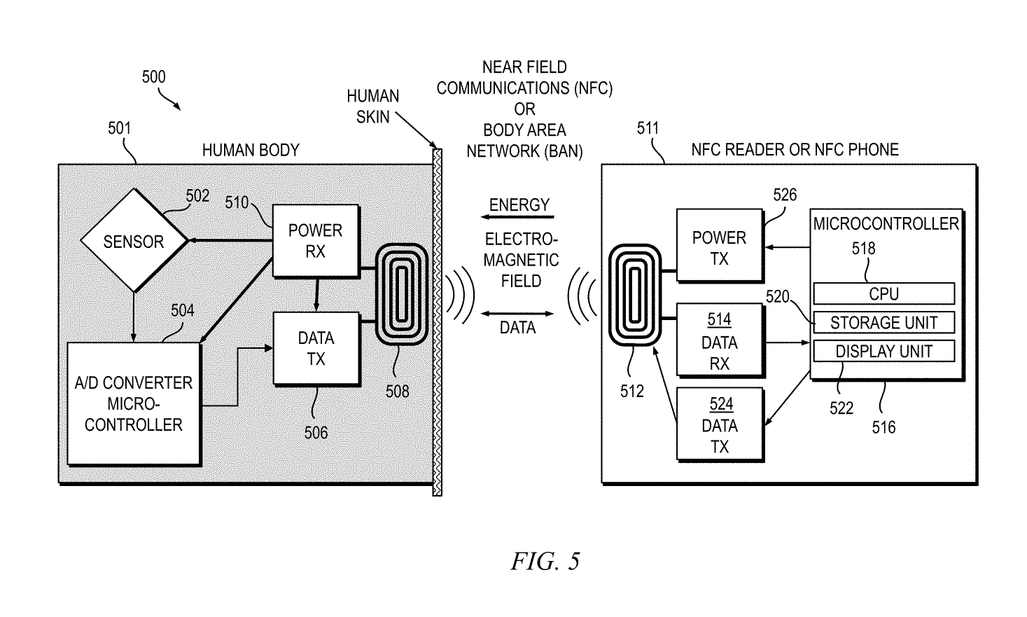

By way of example only and without limitation, FIG. 5 is a schematic diagram depicting at least a portion of an exemplary strain sensor system 500 including wireless transmission capabilities, according to an embodiment of the invention. The sensor system 500 includes a sensor circuit 501 which is adapted for use within the human body. The sensor circuit 501 includes a strain sensor 502, which in one or more embodiments is implemented in a manner consistent with any of the illustrative strain sensor circuits 300, 320 and 350 shown in FIGS. 3A, 3B and 3D, respectively (e.g., including a strain gauge and Wheatstone bridge detector). The strain sensor 502, in one or more embodiments, preferably includes an amplifier for amplifying a sense signal generated by the sensor which is indicative of a strain measurement, as previously described. The sensor circuit 501 further includes an A/D converter circuit 504 adapted to receive the analog sense signal and to generate a digital representation of the sense signal. The A/D converter 504 may include a microcontroller or similar processor configured to format the data for transmission by a data transmitter 506 (Data Tx). The data transmitter 506 is connected to an antenna 508 for transmitting the data indicative of the strain measurement to a corresponding receiving device 511, which may be an NFC reader or phone.

In order to provide power for one or more components in the sensor circuit 501, the circuit preferably includes a power receiver (Power Rx) 510 which is coupled with the antenna 508. The power receiver 510 is configured to receive an energy signal from the antenna 508 which is supplied to the sensor circuit 501 by an external power source residing outside the body, such as using NFC. The power receiver 510 is further configured to extract from the energy signal a supply voltage or current suitable for powering one or more components in the sensor circuit 501 (e.g., sensor 502, A/D converter and microcontroller 504 and data transmitter 506), as indicated using bold arrows. The power receiver 510 may, in one or more embodiments, include rectification circuitry (e.g., a diode) for converting the received energy signal to a direct current (DC) supply voltage. The energy signal may be in the form of an electromagnetic field in close proximity to the embedded sensor circuit 501.

In one or more embodiments, in addition to sending power to the strain sensor system 500, NFC or BAN (body area network) protocols may be used to read data from the sensor circuit 501. NFC constitutes a set of communication protocols that enable two electronic devices, one of which is usually a portable device such as a smartphone, to establish communication by bringing them within close proximity (e.g., about 4-5 cm) to one another. A body area network, also referred to as a wireless body area network (WBAN), is a wireless network of wearable computing devices. BAN devices may be embedded inside the body (e.g., implanted) or may be surface-mounted on the body in a fixed position (e.g., attached to the skin).

The receiving device 511 includes an antenna 512 which is coupled with a data receiver 514. The data receiver 514 is configured to receive data sent by the data transmitter 506 in the sensor circuit 501 from the antenna 512. The received data is supplied to a microcontroller 516, or other microprocessor, which is configured to perform processing of the received data signal. The microcontroller 516, in one or more embodiments, includes a central processing unit (CPU) 518, a storage unit (e.g., memory) 520 coupled with the CPU, and a display unit 522 also coupled with the CPU. The microcontroller 516 may be configured to function in a manner consistent with the processor module 408 shown in FIG. 4. The storage unit 520 may be employed, in one or more embodiments, to collect historical strain measurement data, which can be used for estimating when an implantable device will malfunction, among other features. The receiving device 511 further includes a data transmitter 524 coupled with the microcontroller 516 and the antenna 512. The data transmitter 524 is configured, in one or more embodiments, to receive data (e.g., command and control signals) from the microcontroller 516 and to transmit the data to the sensor circuit 501 via the antenna 512.

The receiving device 511, in one or more embodiments, includes a power transmitter 526 coupled with the microcontroller 516 and the antenna 512. The power transmitter 526 is configured to generate a power signal, which is preferably initiated by a control signal supplied by the microcontroller 516, for transferring power wirelessly to the sensor circuit 501 via the antenna 512, as previously stated. The power signal generated by the power transmitter 526 may employ an NFC protocol, or similar wireless power transmission protocol, configured to supply power to the sensor circuit 501 when placed in close proximity to the sensor circuit (e.g., about 4-5 cm). It is to be understood that embodiments of the invention are not limited to the power transmission methodology used to supply power to the sensor circuit 501.

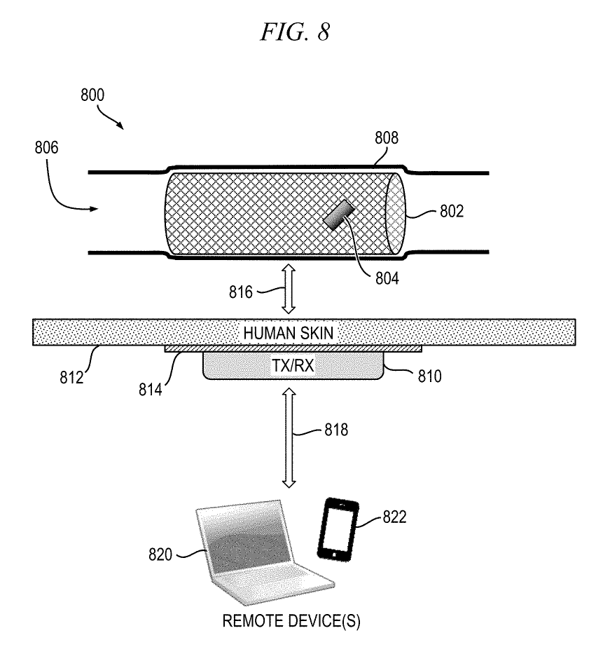

In one or more embodiments, at least one component of the strain sensor system 500 (e.g., the microcontroller 504) is attached to the outside surface of the skin, preferably in close proximity to the strain sensor 502. This embodiment is described further in conjunction with FIG. 8. In this manner, data from the strain sensor, either in analog or digital form, is transmitted wirelessly to the microcontroller 504 for further processing. Such processing may include, for example, determining the flow and/or pressure of bodily fluids or tissue in contact with the sensor directly or indirectly. As previously stated, this information can be used to detect a process compromising the functioning of the implantable device to which the strain gauge is attached, such as, for example, blood clotting, infection, inflammation or tissue proliferation leading to restenosis of the lumen, or mechanical malfunction of the implantable device (e.g., kink or dislocation).

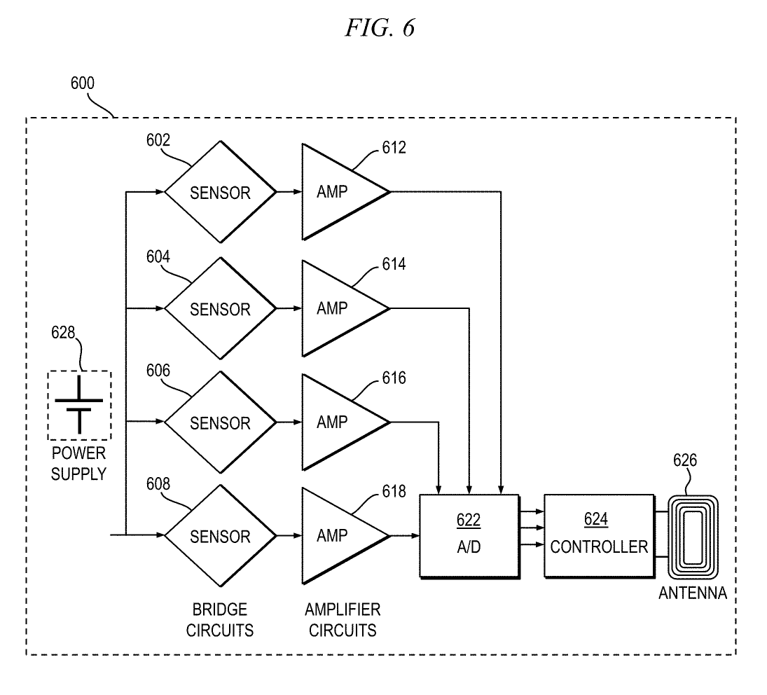

With reference now to FIG. 6, a schematic diagram depicts at least a portion of an exemplary strain sensor system 600 which utilizes four strain gauges and four independent Wheatstone bridge circuits, according to an embodiment of the invention. More particularly, the strain sensor system 600 includes first, second, third and fourth sensor circuits 602, 604, 606 and 608, respectively. Each of the sensor circuits preferably comprises a strain gauge coupled with a resistance bridge, preferably a Wheatstone bridge, or alternative detection circuitry. An output of each of the sensor circuits 602, 604, 606 and 608 is connected to a corresponding amplifier 612, 614, 616 and 618, respectively, for amplifying a sense signal generated by each of the sensor circuits. Each of the sense signals is indicative of a strain measurement generated by each of the strain gauges in the respective sensor circuits. Amplified sense signals generated by the amplifiers 612, 614, 616, 618 are supplied to an A/D converter 622 configured to convert the respective analog sense signals into a digital representation thereof. The digital data indicative of the strain measurements from each of the sensor circuits is then supplied to a communication controller 624, or similar processor, where the data is formatted for transmission via an antenna 626. This multi-sensor embodiment may be useful, for example, in an application in which multiple characteristics and/or multiple locations are being monitored, such as the illustrative application shown in FIG. 3C where two ends of a stent are being monitored simultaneously to determine fluid flow rate, among other factors.

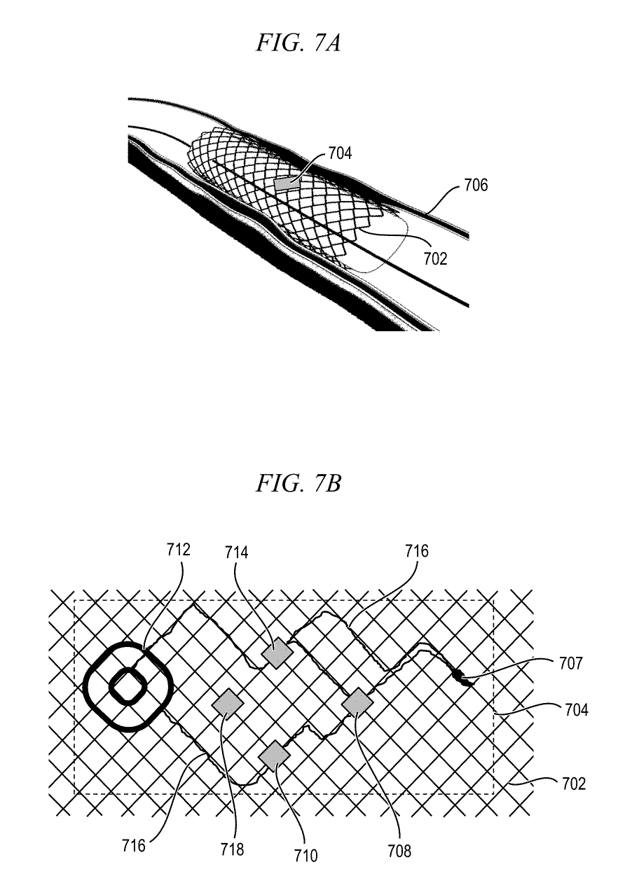

FIGS. 7A and 7B depict an illustrative application of an implantable medical device, in this case, a stent 702, with attached strain sensor 704, according to an embodiment of the invention. In FIG. 7A, the stent 702 and attached strain sensor 704 are shown implanted within the lumen of a coronary artery 706, or alternative lumen through which bodily fluids flow. Flow across the stent 702 is measured using the attached strain sensor 704 in a manner consistent with embodiments of the invention previously described.

FIG. 7B conceptually depicts at least a portion of the illustrative strain sensor circuit 704 attached to the stent 702. With reference to FIG. 7B, the strain sensor circuit 704 includes a strain gauge sensor 707, which may be implemented in a manner consistent with any of the exemplary strain gauge sensors described herein (e.g., strain sensors 300, 320 and 350 shown in FIGS. 3A, 3B and 3D, respectively). In one or more embodiments, at least a portion of the strain gauge and/or Wheatstone bridge is implemented using the conductive mesh forming the stent 702. In one or more embodiments, an I-shaped semiconductor strain gauge is employed (e.g., strain gauge 200 shown in FIG. 2A) and replaces at least a portion of the mesh wire forming the stent 702. Embodiments of the invention are not limited to the shape of the strain gauge; that is, semiconductor strain gauges configured in other shapes (e.g., U-shaped 210, shown in FIG. 2B, or W-shaped 220, shown in FIG. 2C) may also be utilized.

The sensor 707 may include an amplifier integrated therewith for amplifying a sense signal generated as an output of the sensor for subsequent processing or transmission. The strain gauge sensor 707 is electrically coupled with an A/D converter 708, which in one or more embodiments comprises a microcontroller or similar processor integrated therewith. The A/D converter 708 is adapted to receive the analog sensor output signal and to generate a digital sense signal representative of the sensor output signal. This digital sense signal is indicative of the change in resistance of the strain gauge 707 when contacted by bodily fluids flowing through the stent 702; the change in resistance is correlated to strain measurement as previously described. The flow and/or pressure of bodily fluids in contact with the sensor 707 can be extracted from the strain measurement by the microcontroller included within the A/D converter 708.

Alternatively, the data output by the A/D converter 708 can be transmitted to an external processor module (e.g., via a wireless communication link established between the strain sensor 704 and the external processor) for subsequent processing. To accomplish this, the strain sensor 704 further comprises a data transmitter 710 coupled with the A/D converter 708. The data transmitter 710 is configured, in one or more embodiments, to transmit data collected from the A/D converter 708 to a remote device (e.g., smartphone, computer, cloud service, etc.) via an antenna 712 coupled with the transmitter where the information can be further analyzed and compared to other data streams, such as, for example, an electrocardiogram (EKG or ECG) signal. A change in the pattern of flow such as by increased velocity may suggest narrowing of the effective area through which blood or another bodily fluid flows. Occlusion of the stent (e.g., by a thrombus, an embolus, plaque, restenosis, etc.) will result in reduced flow velocity across the stent.

A power receiver 714 included in the strain sensor 704 is coupled with the antenna 712 and one or more components of the strain sensor system and is configured to extract a supply voltage or current from an applied electromagnetic field in close proximity to the device (e.g., using NFC or alternative wireless power technology). Specifically, the receiver 714 is configured to receive an external power signal (e.g., transmitted by an external power source in close proximity to the sensor circuit 704) and to extract therefrom a DC voltage for powering one or more functional components in the sensor circuit 704. The receiver 714 may also be configured to receive commands for controlling an operation of the sensor 704, in one or more embodiments. For example, the receiver may receive start and stop commands to remotely start or end a sensing operation. The functional components in the strain sensor 704 are electrically connected using wire 716, or other conductive structures within the sensor. For instance, the conductive mesh of the stent 702 may be used to electrically connect one or more components of the sensor circuit 704.

In one or more embodiments, a voltage or current used to power one or more functional components in the strain sensor circuit 704 is extracted from the host body in which the implantable medical device is inserted. To accomplish this, the strain sensor 704 may include an energy conversion module configured to generate a voltage or current by harvesting energy generated by the host body. For example, in one or more embodiments, the energy conversion module may comprise a voltage generator which uses heat produced by the body to generate a voltage suitable for powering at least a portion of the functional components in the strain sensor 704. Optionally, in one or more embodiments where no external power is provided, the sensor 704 includes a battery 718, or other power storage element (e.g., storage capacitor), for supplying power to at least a subset of the functional components in the sensor.

FIG. 8 conceptually depicts an illustrative strain sensor system 800 in which one or more components of the system are attached to the outside surface of the skin, according to an embodiment of the invention. With reference to FIG. 8, the system 800 includes a stent 802, or other implantable device, with attached strain sensor 804 implanted within the lumen 806 of a coronary artery 808, or alternative lumen through which bodily fluids flow. Flow across the stent 802 is measured using the attached strain sensor 804 in a manner consistent with embodiments of the invention previously described. The strain sensor 804 is adapted to generate a sense signal indicative of the strain caused by the flow and/or pressure of bodily fluids or tissues in contact with the stent 802.

In this embodiment, one or more components of the strain sensor system 800, such as data transmitter/receiver (TX/RX) circuitry 810, are attached to the outside surface of the skin 812 by way of an adhesive patch 814 (e.g., dermal patch), or alternative attachment means (e.g., an adapter configured to secure the data transmitter/receiver circuitry 810 to the skin 812). A receiver portion of the transmitter/receiver circuitry 810 is configured to receive the sense signal generated by the strain sensor 804 using a short-distance (e.g., less than about 6 cm) wireless communication protocol 816, including, but not limited to, NFC, BAN, RFID, and the like.

The transmitter/receiver circuitry 810, in one or more embodiments, further includes a microcontroller or other processing module, which may be implemented in a manner consistent with the microcontroller 504 shown in FIG. 5 or similar. The microcontroller in the transmitter/receiver circuitry 810 is adapted to process the received sense signal for transmission by a transmitter portion of the transmitter/receiver circuitry. For example, in one or more embodiments, the microcontroller is configured to convert an analog sense signal into a digital representation of the sense signal and to format the digital sense signal into a suitable long-distance (e.g., greater than about 20 feet) wireless communication protocol 818 for transmission to one or more corresponding remote receiving devices, such as, for example, a laptop computer 820, smartphone 822, etc. Suitable long-distance wireless communication protocols include, but are not limited to, Wi-Fi, Bluetooth, ANT+, Worldwide Interoperability for Microwave Access (WiMAX), IEEE 802.11 standards, and the like. Embodiments of the invention are not limited to any specific wireless communication protocols for transmissions between the embedded strain sensor 804 and the transmitter/receiver circuitry 810, or between the transmitter/receiver circuitry and the remote devices 820, 822.

Since NFC communication is limited in transmission distance (e.g., less than about 10 cm), the transmitter/receiver circuitry 810 is preferably attached in close proximity to the strain sensor 804. The transmitter/receiver circuitry 810 preferably includes its own power source, such as a battery, and is therefore capable of supplying the power necessary to support longer-range wireless communication protocols compared to the strain sensor 804.

It is to be appreciated that the various components shown in the accompanying figures may not be drawn to scale. Furthermore, one or more elements of a type commonly used in a practical implementation of the inventive sensor apparatus may not be explicitly shown in a given figure for ease of explanation. This does not imply that the element(s) not explicitly shown are omitted in the actual device.

Given the discussion thus far, it will be appreciated that, in general terms, an exemplary apparatus for monitoring the strain caused by bodily fluids or tissues in contact with an implantable medical device includes at least one strain gauge sensor embedded within the implantable medical device. The strain gauge sensor is configured to measure a mechanical strain of the implantable medical device. The apparatus further includes a processor module operatively coupled with the strain gauge sensor. The processor module is configured to receive a sense signal generated by the strain gauge sensor and to extract therefrom a measurement of strain caused by bodily fluids or tissues in contact with the implantable medical device. In one or more embodiments, the connection between the strain gauge sensor and the processor module is wireless, in which case the apparatus includes a transmitter adapted to send the sense signal to the processor module via a wireless communication protocol.

Given the discussion thus far, it will be appreciated that, in general terms, an exemplary method for monitoring the strain caused by bodily fluids or tissues in contact with an implantable medical device includes: embedding a strain gauge sensor with the implantable medical device, the strain gauge sensor generating a sense signal indicative of a strain caused by bodily fluids or tissues in contact with the implantable medical device; extracting a measurement of the strain caused by bodily fluids or tissues in contact with the implantable medical device from the sense signal generated by the strain gauge sensor; and determining an estimate of a flow of the bodily fluids through the implantable medical device as a function of the measurement of the strain.

At least a portion of the apparatus, methods and system described above may be implemented in an integrated circuit. In forming integrated circuits, identical dies are typically fabricated in a repeated pattern on a surface of a semiconductor wafer. Each die includes a device described herein, and may include other structures and/or circuits. The individual dies are cut or diced from the wafer, then packaged as an integrated circuit. One skilled in the art would know how to dice wafers and package die to produce integrated circuits.

Those skilled in the art will appreciate that the exemplary structures discussed above can be distributed in raw form (i.e., a single wafer having multiple unpackaged chips), as bare dies, in packaged form, or incorporated as parts of intermediate products or end products that benefit from having sensor devices formed in accordance with one or more of the exemplary embodiments.

The illustrations of embodiments described herein are intended to provide a general understanding of the various embodiments, and they are not intended to serve as a complete description of all the elements and/or features of apparatus, methods and systems that might make use of the circuits and techniques described herein. Many other embodiments will become apparent to those skilled in the art given the teachings herein; other embodiments are utilized and derived therefrom, such that structural and logical substitutions and changes can be made without departing from the scope of this disclosure. It should also be noted that, in some alternative implementations, some of the steps of exemplary methods described herein may occur out of the order described or noted in the figures (where shown). For example, two steps described or shown in succession may, in fact, be executed substantially concurrently, or certain steps may sometimes be executed in the reverse order, depending upon the functionality involved. The drawings are also merely representational and are not drawn to scale. Accordingly, the specification and drawings are to be regarded in an illustrative rather than a restrictive sense.

Embodiments are referred to herein, individually and/or collectively, by the term “embodiment” merely for convenience and without intending to limit the scope of this application to any single embodiment or inventive concept if more than one is, in fact, shown. Thus, although specific embodiments have been illustrated and described herein, it should be understood that an arrangement achieving the same purpose can be substituted for the specific embodiment(s) shown; that is, this disclosure is intended to cover any and all adaptations or variations of various embodiments. Combinations of the above embodiments, and other embodiments not specifically described herein, will become apparent to those of skill in the art given the teachings herein.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Terms such as “above” and “below,” where used, are intended to indicate positioning of elements or structures relative to each other as opposed to absolute elevation.

The corresponding structures, materials, acts, and equivalents of any means or step-plus-function elements in the claims below are intended to include any structure, material, or act for performing the function in combination with other claimed elements as specifically claimed. The description of the various embodiments has been presented for purposes of illustration and description, but is not intended to be exhaustive or limited to the forms disclosed. Many modifications and variations will be apparent to those of ordinary skill in the art without departing from the scope and spirit thereof. The embodiments were chosen and described in order to best explain principles and practical applications, and to enable others of ordinary skill in the art to understand the various embodiments with various modifications as are suited to the particular use contemplated.

The abstract is provided to comply with 37 C.F.R. § 1.72(b), which requires an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the appended claims reflect, the claimed subject matter may lie in less than all features of a single embodiment. Thus the following claims are hereby incorporated into the Detailed Description, with each claim standing on its own as separately claimed subject matter.

Given the teachings provided herein, one of ordinary skill in the art will be able to contemplate other implementations and applications of the techniques and disclosed embodiments. Although illustrative embodiments have been described herein with reference to the accompanying drawings, it is to be understood that illustrative embodiments are not limited to those precise embodiments, and that various other changes and modifications are made therein by one skilled in the art without departing from the scope of the appended claims.

What is claimed is:

1. An apparatus for monitoring strain caused by bodily fluids or tissues in contact with an implantable medical device, the apparatus comprising:
at least a first strain gauge sensor embedded within the implantable medical device and configured to measure a mechanical strain of the implantable medical device; and
a processor module operatively coupled with the first strain gauge sensor, the processor module being configured to receive a sense signal generated by the first strain gauge sensor and to extract therefrom a measurement of strain caused by bodily fluids or tissues in contact with the implantable medical device.

2. The apparatus of claim 1, further comprising an analog-to-digital converter coupled between an output of the first strain gauge sensor and an input of the processor module, the analog-to-digital converter being configured to receive the sense signal and to generate a digital representation of the sense signal for subsequent processing by the processor module.

3. The apparatus of claim 1, further comprising an amplifier configured to receive the sense signal generated by the first strain gauge sensor and to generate an amplified sense signal as an output thereof, the amplified sense signal being supplied to the processor module for subsequent processing.

4. The apparatus of claim 1, further comprising a transmitter coupled with the first strain gauge sensor, the transmitter being configured to transmit the sense signal wirelessly to an external processor for processing the sense signal.

5. The apparatus of claim 4, wherein the transmitter is configured to transmit the sense signal using at least one of near-field communication, radio frequency identification, and body area network communication protocols.

6. The apparatus of claim 1, further comprising a power receiver and an antenna coupled with the power receiver, the power receiver being configured to receive an energy signal from an external power source placed in close proximity to the apparatus and to extract from the energy signal at least one of a supply voltage and a supply current for powering at least one of the first strain gauge sensor and the processor module.

7. The apparatus of claim 1, further comprising a second strain gauge sensor embedded within the implantable medical device, wherein the first and second strain gauge sensors are placed at different locations on the implantable medical device for monitoring a flow rate of bodily fluids passing through the implantable medical device.

8. The apparatus of claim 1, wherein the first strain gauge sensor is inserted with the implantable medical device within a mammal body and at least a portion of the processor module is attached to an outside surface of skin of the mammal body proximate the first strain gauge sensor, and wherein the processor module is configured to communicate with the first strain gauge sensor via a wireless communication protocol.

9. The apparatus of claim 8, wherein the portion of the processor module attached to the skin further includes a transmitter for transmitting strain measurement data to a remote receiving device for subsequent processing.

10. The apparatus of claim 9, wherein the transmitter is configured to transmit the strain measurement data using at least one of Wi-Fi, Bluetooth, ANT+, Worldwide Interoperability for Microwave Access (WiMAX) and IEEE 802.11 communication protocols.

11. The apparatus of claim 9, wherein portion of the processor module is attached to the skin using a dermal patch.

12. The apparatus of claim 1, wherein the processor module is embedded within the implantable medical device.

13. The apparatus of claim 1, further comprising a battery embedded within the implantable medical device, the battery supplying power to at least one of the strain gauge sensor and the processor module.

14. The apparatus of claim 1, wherein the strain gauge sensor comprises a semiconductor strain gauge and forms at least a portion of the implantable medical device.

15. The apparatus of claim 1, further comprising a power receiver, the power receiver being configured to receive an energy signal from an external power source and to extract from the energy signal at least one of a supply voltage and a supply current operative to power at least one component in the apparatus.

16. The apparatus of claim 1, wherein the first strain gauge sensor comprises:
at least a first strain gauge; and
a resistance bridge electrically coupled with the first strain gauge, the resistance bridge being configured to generate the output sense signal which changes as a function of variations in a resistance of the first strain gauge caused by bodily fluids or tissues in contact with the implantable medical device.

17. The apparatus of claim 16, wherein the first strain gauge sensor further comprises a second strain gauge coupled with the resistance bridge, the second strain gauge being oriented in a different direction relative to the first strain gauge for measuring strain in multiple axes.

18. A method for monitoring strain caused by bodily fluids or tissues in contact with an implantable medical device and for detecting a malfunction of the implantable medical device leading to impaired flow, the method comprising:
embedding at least a first strain gauge sensor with the implantable medical device, the first strain gauge sensor generating a sense signal indicative of a strain caused by bodily fluids or tissues in contact with the implantable medical device;
extracting a measurement of the strain caused by bodily fluids or tissues in contact with the implantable medical device from the sense signal generated by the first strain gauge sensor; and
determining an estimate of a flow of the bodily fluids through the implantable medical device as a function of the measurement of the strain.

19. The method of claim 18, further comprising transmitting the sense signal wirelessly from the first strain gauge sensor to a processor module for determining the estimate of the flow of bodily fluids as a function of the sense signal.

20. The method of claim 18, further comprising forming at least a portion of the implantable medical device using the first strain gauge sensor.
